(19) United States
(12) Patent Application Publication
(10) Pub. No.: US 2023/0328040 A1
(43) Pub. Date: Oct. 12, 2023

(54) COMPLIANT INSIGHT FORUM AND ANALYSIS FOR HEALTHCARE

(71) Applicant: OMNY, Inc., Atlanta, GA (US)

(72) Inventors: Sean O'Brien, Atlanta, GA (US); Stella Young-Chin Chang, Vienna, VA (US); Maik Lindner, Roswell (DE)

(21) Appl. No.: 18/132,271

(22) Filed: Apr. 7, 2023

Related U.S. Application Data
(60) Provisional application No. 63/329,788, filed on Apr. 11, 2022.

(51) Int. Cl. H04L 9/40; G16H 80/00

(52) U.S. Cl. CPC H04L 63/0421; G16H 80/00; H04L 63/20

(57) ABSTRACT

A system for compliant interaction between parties may include a computing device hosting a compliance and insight platform executing on the computing device. The platform may include a secure room hosted on the platform. The secure room may include a restricted environment that hosts interaction data between users based on one or more interactions rules of the secure room. The secure room may be configured to receive interaction data from a first user in the secure room, provide at least a portion of the interaction data to a second user in the secure room, and blind and restrict the first user and the second user from each other's identity. The platform may further include a moderator of the secure room. The moderator may be configured to moderate the interaction data in the first secure room.

### CROSS-REFERENCES TO RELATED APPLICATIONS

This application claims priority to U.S. Provisional Patent Application No. 63/329,788, entitled “COMPLIANT INSIGHT FORUM AND ANALYSIS FOR HEALTHCARE,” filed on Apr. 11, 2022, which is pending, and the entire contents of this application are incorporated herein by reference.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### STATEMENT REGARDING FEDERALLY SPONSORED RESEARCH OR DEVELOPMENT

Not Applicable

### BACKGROUND OF THE DISCLOSURE

The present disclosure generally relates to healthcare technology, and more particularly to a compliant insight forum and analysis for healthcare.

Timely and accurate healthcare data helps advance efficiency in patient care and in drug and medical device development. However, there often exist regulatory or ethical barriers for healthcare providers and healthcare companies (whether a pharmaceutical company, medical device company, or some other type of healthcare company) to share healthcare data between them. As an example, a conventional communication setup for pharmaceuticals and healthcare providers may include obtaining approval and process oversight from multiple parties within life sciences sponsor and healthcare provider organizations. The collaboration agreement may be drafted and executed on a case-by-case basis. Legal departments, as well as ethical committees on the healthcare provider side, may determine or affirm that the collaboration process and patterns are ethical, compliant, and legal. Implementation of the collaboration agreement may include engagement beyond legal, business, and compliance. Implementation may include business operations or information technology departments from the life sciences company and the healthcare provider organization. Based on a secure and tracked setup, a sequential collaboration on data and insights may be enabled. To help with consistent compliance, questions, comments, or discussion may be processed by the approval and process oversight body. This may include a time-consuming and often time prohibitive process in today's fast-paced healthcare environment. Speed of data may be important, and this conventional collaboration setup between pharmaceutical companies and healthcare providers may not fulfill the need of modern collaboration and analytics. Furthermore, from an audit perspective, this individual setup of collaboration may post a tremendous burden on manual logging and tracking of collaboration inside data artifacts. In the event of an audit, both sides (the pharmaceutical company as well as the healthcare provider) may need to gather evidence that can be distributed across file shares, emails, or instant messenger communication.

Additionally, current technical frameworks and communication implementations do not address these deficiencies in the healthcare space. Regulated communication is mostly designed on a case-by-case basis. The need for a standardized, commoditized framework is apparent. The current speed with which communication between entities in the healthcare industry does not fulfill the need for close to real-time data consumption, analyses, and decision making; nor is there enough assurance that communication is compliant with legal and information technology security standards. The slow pace that comes with this complex interaction of entities holds back the improvise in the healthcare field.

What is needed, then, are systems and methods for a compliant insight forum and analysis for healthcare.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

The compliant insight forum and analysis enablement for healthcare disclosed herein addresses the current needs and overcomes the current design and technical hurdles of existing approaches. The secure room implementation, as part of a compliant insight forum, may allow for compliant and legal interaction, exchange, and collaboration between life sciences and healthcare providers without violating regulatory and ethical barriers. The implementation of a secure room may be significantly faster than the traditional set up of communication channels between regulated partners and collaborators. The audit function may allow for a case basis review of pre-filtered logs and reports to serve as evidence after the compliant and legal interactions in the forum.

One aspect of the disclosure is a system for compliant interaction between parties. The system may include a computing device hosting a compliance and insight platform executing on the computing device. The platform may include a secure room hosted on the platform. The secure room may include a restricted environment that hosts interaction data between users based on one or more interactions rules of the secure room. The secure room may be configured to receive interaction data from a first user in the secure room, provide at least a portion of the interaction data to a second user in the secure room, and blind and restrict the first user and the second user from each other's identity. The platform may further include a moderator of the secure room. The moderator may be configured to moderate the interaction data in the first secure room.

Another aspect of the disclosure includes a computer-implemented method for compliant interaction between parties in a secure room implemented on a compliance and insight platform. The method may include receiving, at the platform, a compliance template. The compliance template may include data indicating one or more entities, functionality of the secure room, and interaction rules of the secure room. The method may include generating, on the platform, a technical setup based on the compliance template. The technical setup may include computer-executable instructions configured to enforce the interaction rules in the secure room. The method may include providing the secure room based on the interactions rules of the secure room. The method may include permitting a first user and a second user of the platform to interact with one another in the secure room. The first user and the second user interacting in the secure room may include the first user sending interaction data to the secure room, and the platform making at least a portion of the interaction data viewable by the second user. The method may include recording the interaction data on a distributed ledger.

Another aspect of the disclosure includes a non-transitory computer-readable storage medium having a first set of computer-executable instructions stored thereon. In response to a computer processor executing the computer-executable instructions, the computer processor may be configured to (1) receive, at a compliance and insight platform, a compliance template for a secure room, wherein the compliance template may include data indicating one or more entities, functionality of the secure room, and interaction rules of the secure room; (2) generate, on the platform, a technical setup based on the compliance template, wherein the technical setup may include a second set of computer-executable instructions configured to enforce the interaction rules in the secure room; (3) provide the secure room based on the interaction rules of the secure room; (4) permit a first user and a second user of the platform to interact with one another in the secure room, wherein the first user and the second user interacting in the secure room includes (a) the first user providing a dataset to the secure room, (b) the platform making at least a portion of the dataset downloadable from the platform by the second user, (c) the second user sending text-based communication data to the secure room, and (d) the platform making at least a portion of the text-based communication data viewable in the secure room by the first user; and (5) record the text-based communication data on a distributed ledger.

The systems and methods disclosed herein provide several technical improvements to computing devices and over conventional compliance systems. First, by recording interaction data, attestation data, and other data of the compliance and insight platform on a distributed ledger, such data can be recorded immutably as to provide a record that is not siloed in a private entity's data storage and is not changeable by the that private entity. Second, the secure room provided by the platform provides technological ways of communication (e.g., instant text chat, audio chat, video chat) that are moderated in real time by moderators (including artificial intelligence moderators) in order to remain compliant. Third, the platform can reuse compliance templates and technical setups, modifying them slightly in response to different compliance requirements, in order to quickly generate new secure rooms. Numerous other objects, advantages and features of the present disclosure will be readily apparent to those of skill in the art upon a review of the following drawings and description of various embodiments.

### BRIEF DESCRIPTION OF THE DRAWINGS

FIG. 1 is a schematic block diagram illustrating one embodiment of a system for a compliant insight forum and analysis for healthcare.

FIG. 2 is a schematic block diagram illustrating one embodiment of a compliance template for use in a compliant insight forum and analysis for healthcare.

FIG. 3 is a schematic block diagram illustrating one embodiment of a technical setup for use in a compliant insight forum and analysis for healthcare.

FIG. 4 is a flowchart illustrating one embodiment of generating a secure room for use in a compliant insight forum and analysis for healthcare.

FIG. 5 is a schematic block diagram illustrating one embodiment of a user joining one or more secure rooms using a platform for an compliant insight forum and analysis for healthcare.

FIG. 6 is a schematic block diagram illustrating one embodiment of an entity undergoing an attestation process in order to join a secure room in a compliant insight forum and analysis for healthcare.

FIG. 7 is a flowchart diagram illustrating one embodiment of a method for a compliant insight forum and analysis for healthcare.

### DETAILED DESCRIPTION

While the making and using of various embodiments of the present disclosure are discussed in detail below, it should be appreciated that the present disclosure provides many applicable inventive concepts that are embodied in a wide variety of specific contexts. The specific embodiments discussed herein are merely illustrative of specific ways to make and use the disclosure and do not delimit the scope of the disclosure. Those of ordinary skill in the art will recognize numerous equivalents to the specific apparatus and methods described herein. Such equivalents are considered to be within the scope of this disclosure and are covered by the claims.

In the drawings, not all reference numbers are included in each drawing, for the sake of clarity. In addition, positional terms such as “upper,” “lower,” “side,” “top,” “bottom,” etc. refer to the apparatus when in the orientation shown in the drawing. A person of skill in the art will recognize that the apparatus can assume different orientations when in use.

Reference throughout this specification to “one embodiment,” “an embodiment,” “another embodiment,” or similar language means that a particular feature, structure, or characteristic described in connection with the embodiment is included in at least one embodiment. Thus, appearances of the phrases “in one embodiment,” “in an embodiment,” “in some embodiments,” and similar language throughout this specification may, but do not necessarily, all refer to the same embodiment, but mean “one or more but not necessarily all embodiments” unless expressly specified otherwise.

The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. As used herein, the term “a,” “an,” or “the” means “one or more” unless otherwise specified. The term “or” means “and/or” unless otherwise specified.

Multiple elements of the same or a similar type may be referred to as “Elements 102(1)-(n)” where n may include a number. Referring to one of the elements as “Element 102” refers to any single element of the Elements 102(1)-(n). Additionally, referring to different elements “First Elements 102(1)-(n)” and “Second Elements 104(1)-(n)” does not necessarily mean that there must be the same number of First Elements as Second Elements and is equivalent to “First Elements 102(1)-(n)” and “Second Elements (1)-(m)” where m is a number that may be the same or may be a different number than n.

As used herein, the term “computing device” may include a desktop computer, a laptop computer, a tablet computer, a mobile device such as a mobile phone or a smart phone, a smartwatch, a gaming console, an application server, a database server, or some other type of computing device. A computing device may include a physical computing device or may include a virtual machine (VM) executing on another computing device. A computing device may include a cloud computing system, a distributed computing system, or another type of multi-device system.

As used herein, the term “data network” may include a local area network (LAN), wide area network (WAN), the Internet, or some other network. A data network may include one or more routers, switches, repeaters, hubs, cables, or other data communication components. A data network may include a wired connection or a wireless connection.

As used herein, the term “computing platform” or “platform” may include a computing environment where a portion of software can execute. A computing platform may include hardware on which the software may execute. The computing platform may include an operating system. The computing platform may include one or more software applications, scripts, functions, or other software. The computing platform may include one or more application programming interfaces (APIs) by which different portions of the software of the platform may communicate with each other or invoke functions. The computing platform may include one or more APIs by which it may communicate with external software applications or by which external software applications may interact with the platform. The computing platform may include a software framework. The computing platform may include one or more VMs. The software platform may include one or more data storages. The software platform may include a client application that executes on an external computing device and that interacts with the platform in a client-server architecture.

As used herein, the term “data storage” may include a tangible device that retains and stores data. Such device may include an electronic storage device, a magnetic storage device, an optical storage device, an electromagnetic storage device, a semiconductor storage device, or any suitable combination of the foregoing. A non-exhaustive list of more specific examples of the devices may include a portable computer diskette, a random access memory (RAM), a read-only memory (ROM), an erasable programmable read-only memory (EPROM or flash memory), a static random access memory (SRAM), a hard disk drive (HDD), a solid state drive, a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. “Data storage,” in some embodiments, may include a data structure that stores data, and the data structure may be stored on a tangible data storage. Such data storage may include a file system, a database, cloud storage, a data warehouse, a data lake, or other data structures configured to store data.

As used herein, the terms “determine” or “determining” may include a variety of actions. For example, “determining” may include calculating, computing, processing, deriving, looking up (e.g., looking up in a table, a database or another data structure), ascertaining, or other actions. Also, “determining” may include receiving (e.g., receiving information or data), accessing (e.g., accessing data in a memory, data storage, distributed ledger, or over a network), or other actions. Also, “determining” may include resolving, selecting, choosing, establishing, or other similar actions.

As used herein, the terms “provide” or “providing” may include a variety of actions. For example, “providing” may include generating data, storing data in a location for later retrieval, transmitting data directly to a recipient, transmitting or storing a reference to data, or other actions. “Providing” may also include encoding, decoding, encrypting, decrypting, validating, verifying, or other actions.

As used herein, the term “access,” “accessing”, and other similar terms may include a variety of actions. For example, accessing data may include obtaining the data, examining the data, or retrieving the data. Providing access or providing data access may include providing confidentiality, integrity, or availability regarding the data.

As used herein, the term “message” may include one or more formats for communicating (e.g., transmitting or receiving) information or data. A message may include a machine-readable collection of information such as an Extensible Markup Language (XML) document, fixed-field message, comma-separated message, or another format. A message may, in some implementations, include a signal utilized to transmit one or more representations of information or data.

As used herein, the term “user interface” (also referred to as an interactive user interface, a graphical user interface or a UI), may refer to a computer-provided interface including data fields or other controls for receiving input signals or providing electronic information or for providing information to a user in response to received input signals. A user interface may be implemented, in whole or in part, using technologies such as hyper-text mark-up language (HTML), a programming language, web services, or rich site summary (RSS). In some implementations, a user interface may be included in a stand-alone client software application configured to communicate in accordance with one or more of the aspects described.

As used herein, the term “modify” or “modifying” may include several actions. For example, modifying data may include adding additional data or changing the already-existing data. As used herein, the term “obtain” or “obtaining” may also include several types of action. For example, obtaining data may include receiving data, generating data, designating data as a logical object, or other actions.

As used herein, the term “data object” may include a logical container for data. A data object may include an instance of an object in a software application implemented with an object-oriented programming language. A data object may include data formatted in an electronic data interchange (EDI) format, such as an eXtensible Markup Language (XML) object, a JavaScript Object Notation (JSON) object, or some other EDI-formatted object. A data object may include one or more functions that may manipulate the data of the data object. For example, a data object may include the functions or methods of an object in a software application implemented with an object-oriented programming language.

As used herein, the term “decentralized” means that at least a portion of information or functionality is not controlled by a single party. Instead the decentralized information or functionality is distributed among several parties whose aggregate behavior affects the information or functionality. One example of a decentralized technology is a distributed ledger.

As used herein, the term “distributed ledger” may include a data storage of transactions replicated across and synchronized by multiple computers, called “nodes,” in communication with each other. The nodes may synchronize the data of the distributed ledger, including which transactions are added to the ledger and in what order, using a consensus mechanism. The transactions may be cryptographically secured such that once a transaction is added to the distributed ledger, the transaction cannot be later modified. Basic encryption or cryptography principles—such a public key infrastructure, digital signatures, and other cryptographic technologies—underlie the application of distributed ledger technology. When a user adds a distributed ledger transaction, the user may digitally sign the transaction such that other parties can verify that the transaction did, in fact, originate from that user. One example implementation of a distributed ledger includes a blockchain.

As used herein, the term “blockchain” may include a list of blocks that are cryptographically linked together. Each block may include one or more blockchain transactions. Each block may include a cryptographic hash of the previous block in the blockchain. Each block may include a timestamp, which may include the timestamp of when the block was generated or when the block was added to the blockchain. The blockchain may be maintained as replicated and synchronized copies across a blockchain network of nodes. The nodes may generate blocks and may determine which transactions are included in which blocks and in what order, and the nodes may synchronize their blockchain copies via a consensus mechanism.

As used herein, a “token” may include a data asset represented by data on a blockchain. A token may be transferable between blockchain users via a transaction from a first user to a second user. As used herein, a “tokenized” asset may refer to an asset whose ownership interest is represented by one or more tokens. Thus, a user who owns a token may own at least a portion of the corresponding asset. A token representing a tokenized asset may directly represent ownership the asset or indirectly represent ownership of the asset. A token directly representing ownership may include the token itself representing ownership of the asset. A token indirectly representing ownership of the asset may include the token representing ownership of some other thing, which in turn may represent ownership of the asset. As an example of a token indirectly representing ownership of an asset, a token may represent ownership of at least part of a business entity such as a corporation, and the corporation may own a piece of real estate property. The token represents ownership of the piece of real estate property even though it is through ownership of the business entity.

As used herein, the term “transaction” may include a blockchain transaction. A blockchain transaction may include a source address, a destination address, an amount of cryptocurrency or a token, a timestamp indicating when the transaction was generated or other data. As used herein, a “cryptocurrency wallet” may include data that may allow a user of a blockchain network to send or receive cryptocurrency or tokens via the blockchain network. The cryptocurrency wallet may include one or more public keys, private keys, or other cryptographic components used to generate transactions. A cryptocurrency wallet may include a corresponding cryptocurrency wallet address that may uniquely identify the cryptocurrency wallet. As used herein, the terms a first user “sending” a second user cryptocurrency or a token may include the first user generating a transactions where the destination address is the cryptocurrency wallet address of the second user. Hence, using a cryptocurrency wallet may not mean that cryptocurrency or a token is physically sent from, received at, or held in a cryptocurrency wallet, but, instead, transactions of the blockchain network indicate that the owner of the cryptocurrency wallet has ownership of certain cryptocurrency or tokens.

As an overview, many patient-healthcare provider encounters (e.g., patient visits to a doctor office) generate records that can be considered raw data. Such records may include electronic health records (EHR), insurance claims, office or hospital administrative records, or other types of records. The raw data may include important information for healthcare companies looking to develop healthcare products (e.g., a new pharmaceutical or medical device), but the raw data may be unsuitable and may have low value due to the data being unstructured or in a variety of disparate formats. For a healthcare company to gain insights for new products, treatments, and better patient outcomes, the raw data may need cleaning, normalizing, and may need to be structured into usable data packages. These useful units are referred to herein as “data products.” Based on these data products, data scientists can build models to analyze and derive insights. Cleaning and analysis may unlock insights from the raw data.

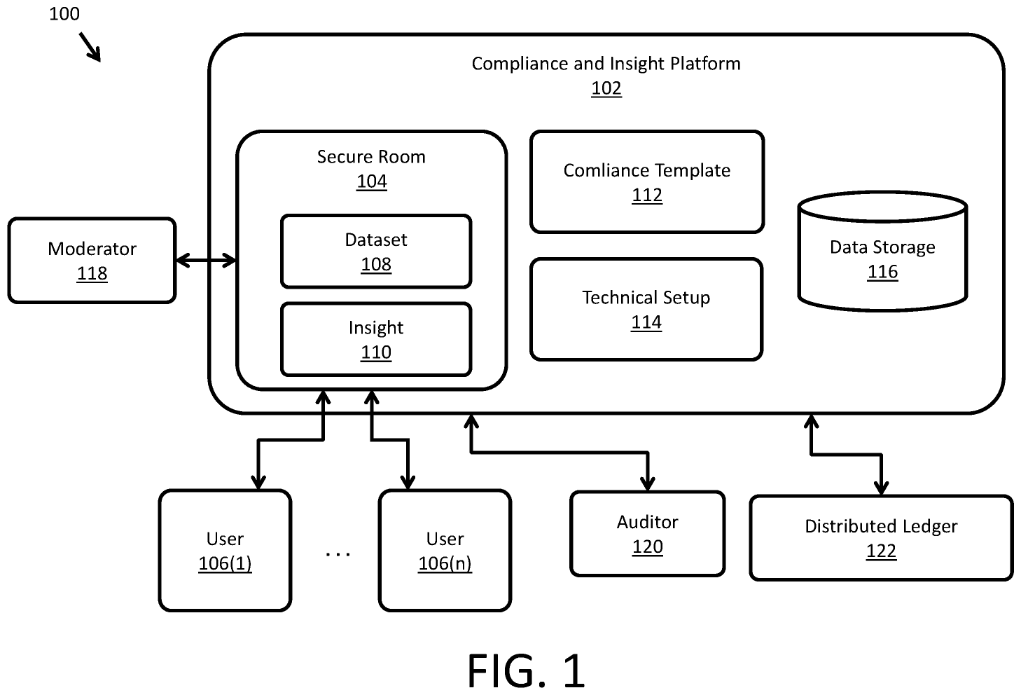

FIG. 1 depicts one embodiment of a system 100. The system 100 may include a system for compliant insight forum and analysis for healthcare. As shown in FIG. 1, the system 100 may include one or more system architecture components. These system architecture components may be utilized for the technical implementation of a compliance setup and may fulfill the need for compliant and secure interactions between users.

As an overview, the system 100 may include a compliance and insight platform 102 (sometimes referred to as “the platform 102”). The platform 102 may be hosted on a computing device. The platform 102 may include hardware and software that provides the systems and methods for a compliant insight forum and analysis for healthcare. The platform 102 may carry out the functionality of such systems and methods.

In one embodiment, the platform 102 may include or host a secure room 104. The secure room 104 may include a logical collaboration space between participating users 106 of the platform 102. The secure room 104 may include a restricted environment that hosts interaction data between users 106 of the platform. The secure room 104 may host the interaction data based on one or more interactions rules of the secure room 104. The secure room 104 may support various forms of interactions and communications between the users 106(1)-(n), such as email, text chat, voice chat, or video communication. In some embodiments, the interactions between the users 106 in the secure room 104 may include a user 106(1) making available one or more datasets 108 or one or more insights 110 for another user 106(2) to view, access, or download. The platform 102 may include one or more compliance templates 112. A compliance template 112 may include data that indicates how a certain secure room 104 functions such that the interactions in the secure room 104 are compliant with certain governmental and regulatory rules related to the exchange of healthcare data. The platform 102 may include one or more technical setups 114. A technical setup 114 may include one or more data objects that may implement one or more of the components of a compliance template 112 to set up a secure room 104. In some embodiments, the platform 102 may include a data storage 116. The data storage 116 may store at least a portion of the data used by the platform 102.

In some embodiments, the system 100 may include one or more users 106. A user 106 may include a person or other entity that uses the platform 102. A user 106 may include a pharmaceutical company, a medical device company, a healthcare provider, a doctor office, a hospital, or some other entity. A user 106 may have an account on the platform 102. A user 106 may interact with different components of the platform 102 and perform various function on the platform 102 using the user's 106 corresponding account. The user's 106 account on the platform 102 may include data associated with the user 106.

The system 100 may include a moderator 118. A moderator 118 may include a person, piece of software of the platform 102, a user 106, or some other entity that may help ensure compliance in the secure room 104 or may respond to violations of a rule of a secure room 104. The system 100 may include an auditor 120. An auditor 120 may include a person, government entity, user 106, or other entity that may audit a portion of the platform 102 for compliance. In one embodiment, the system 100 may include a distributed ledger 122. The distributed ledger 122 may capture one or more relevant events that may occur on or related to the platform 102.

Further details of the components of the system 100 of FIG. 1 are now discussed. The platform 102 may include one or more secure rooms 104. As mentioned above, a secure room 104 may include a logical collaboration space between participating users 106 of the platform 102. The secure room 104 may support various forms of interactions between the users 106(1)-(n). The secure room 104 may blind and restrict users 106 from each other's identity in order to be compliant.

In some embodiments, the secure room 104 may include interaction data. The interaction data may include data reflecting interactions in the secure room 104 between users 106. The secure room 104 may receive interaction data from a user 106(1). The secure room 104 may provide at least a portion of the data to one or more other users 106(2)-(n). The interaction data may include communication data. Communication data may include email data (whether internal to the platform 102 or using an external, third-party email service), text chat data, voice chat data, or video chat data (which may include video or audio). Communication data may include one-on-one user 106 communication or communication from one user 106 to multiple users 106. Interaction data may include a user 106 sending files or other data to another user 106. Such files may include a dataset 108, insights 110, or other data. Interaction between users 106 may include other interaction activities.

In one embodiment, a user 106 may log onto the platform 102 via a website, a software application on the user's 106 computing device, a mobile application on the user's mobile device, or via some other method. The user 106 may select a secure room 104 to enter from a list of secure rooms 104 available to the user 106. The secure room 104 may be represented as a user interface on the user's 106 computing device. The user interface may include a text chat area where text chat messages can be sent and displayed. The user interface may include an area where files can be uploaded or downloaded. The user interface may include a video communication area where the user 106 may view a video feed from another user 106. In one embodiment, the video feed of a user 106 may include a virtual avatar such that the identity of the user 106 is not known by one or more other users 106. The audio of the video feed may be altered such that one or more other users 106 cannot identify the originating user 106 (e.g. using pitch shifting). The user interface may include an area where the user 106 can interact to send audio data to or receive audio data from another user 106. In some embodiments, the platform 102 may adjust the audio data such that one or more other users 106 cannot identify the originating user 106. In some embodiments, role or permission-based access to the secure room 104 by external secure messaging or email services may allow for integration with existing communication technologies via secured channels. The setup of the secure room 104 may assist with compliance of the collaboration and communication happening between participating users 106.

In one or more embodiments, the secure room 104 may include one or more datasets 108. A dataset 108 may be in various formats. A dataset 108 may include healthcare data associated with one or more patients; data associated with one or more diseases or conditions; data associated with a virus, bacteria, or other organisms; research data in the healthcare field; or other healthcare data. The secure room 104 may include one or more insights 110. An insight 110 may be based on one or more of the datasets 108. An insight 110 may include a correlation coefficient showing that patients with certain lab values like BMI respond poorly to corticosteroids.

In some embodiments, the platform 102 may tag certain content associated with the secure room 104. The content may include interaction data. Tagging the content may include associating the content with metadata. The platform 102 may use the metadata for compliance, auditing, searching, or other purposes. The metadata may identify when the content was generated on or provided to the platform 102 or in the secure room 104. The metadata may identify one or more users 106 that generated or provided the content. The metadata may identify one or more users 106 that received, viewed, or downloaded the content. The metadata may identify when the content was received, viewed, or downloaded by such user(s) 106. The metadata may include a confidentiality level. The confidentiality level may include data indicating which users 106, groups of users 106, auditors 120, or other entities may view the tagged content.

In some embodiments, the platform 102 may tag interaction data from the secure room 104. For example, the platform 102 may tag a text chat message as being sent by a certain user 106 at the time the user submitted the message to the secure room 104. The platform 102 may tag a dataset 108 or insight 110. In some embodiments, which content the platform 102 tags may be based on one or more interaction rules of the secure room 104, the compliance template 112 or technical setup 114 of the secure room 104, or other data on the platform 102.

As mentioned previously, a user 106 may have an account on the platform 102. The user's 106 account data may include data about the user. The user's 106 account data may indicate the user's 106 role. A user's role may include a pharmaceutical company, a medical device company, a doctor office, a hospital, a healthcare provider, a pharmaceutical market access user, a clinical provider with formulary discretion, a clinical contract manager with no formulary influence, or some other type of role.

In some embodiments, only a user 106 that has undergone an attestation process may be allowed to participate in a secure room 104. In some embodiments, the attestation function may allow for a systematic screening and vetting of users that may want to collaborate in a secure room 104 in a compliant fashion. The function may help secure the authenticity and correctness of user 106 setup in the communication and collaboration environment. Attestation may include strict language crafted by legal experts that may provide confirmation that no statutes will be violated. In response to a user 106 digitally signing a document committing to a legal and ethical path, the digital signature may be placed on the distributed ledger 122.

In one embodiment, a moderator 118 may moderate the interaction data in the secure room 104. A moderator 118 may include a human or make include a piece of software (e.g., an artificial intelligence (AI) model). A software moderator may include rule-based support. A moderator 118 may moderate compliance of collaboration and communication over datasets 108, insights 110, and communication in a secure room 104. In response to a user 106 violating an interaction rule of the secure room 104, a moderator 118 may perform a corrective action. A corrective action may include a correction to or replacement of text or audio in interaction data, restriction of other communication efforts by a user 106, a potential warning to the violating user 106, the exclusion of the violating user 106 from the secure room 104, or other actions. In one embodiment, a moderator 118 may include a person that is an employee of the entity that owns, operates, or controls the platform 102. In some embodiments, the moderator 118 may include a third-party entity (i.e., is not employed or otherwise associated with the entity that owns, operators, or controls the platform 102). In some embodiments, the moderator 118 may include a user 106 of the platform 102 that has been granted moderator access or privileges by the platform 102.

The system 100 may include one or more auditors 120. An auditor 120 may include a governmental agency or employee thereof or some other entity that has an interest in auditing the platform 102. An audit function of the system 102 may allow an auditor 120 to register with the platform 102 and review data and events associated with the platform 102. Such auditable data may include interaction data, data associated with a user 106 or its account on the platform 102, data related to an attestation associated with a user 106, datasets 108, insights 110, or data associated with interactions between users 106 in the secure room 104.

In some embodiments, data an auditor 120 may audit may be stored in a data storage 116 of the platform 102. The data storage 116 may be physically stored on a computing device of the platform 102. The data storage 116 may be physically stored on a computing device external from the platform 102 but in data communication with the platform 102. In one embodiment, data an auditor 120 may audit may be stored on a shared distributed ledger 120. The distributed ledger 120 may capture one or more events on the platform 102. An event may include a user 106, moderator 118, or auditor 120 registering with the platform 102, interactions between users 106 of the platform 102 (e.g., interactions in a secure room 104), or publicized data or information sets. In some embodiments, data provided to the auditor 120 during an audit may be filtered by the platform 102 such that only data relevant to the audit is visible to the auditor 120. In some embodiments, the auditor 120 may undergo an attestation process. This may result in the auditor 120 being required to adhere to the legal and ethical framework of a secure room 104.

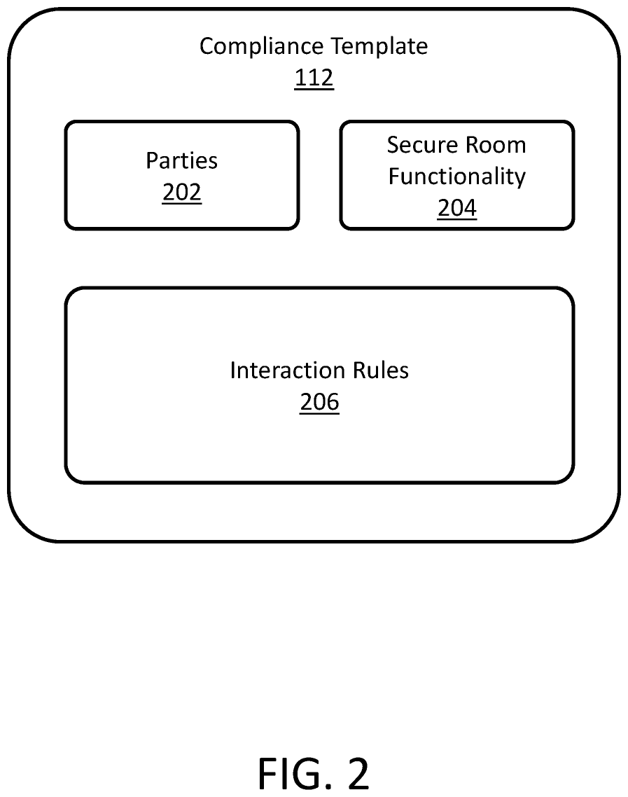

In one embodiment, the platform 102 may execute the functionality of a secure room 104 based on one or more compliance templates 112. A compliance template 112 may include data that indicates how a certain secure room 104 functions such that the interactions in the secure room 104 are compliant with certain governmental and regulatory rules related to the exchange of healthcare data. A compliance template 112 may be reusable such that the platform 102 can set up different secure rooms 104 with the same or similar interaction functionality. FIG. 2 depicts one embodiment of a compliance template 112.

In one embodiment, a compliance template 112 may include data indicating one or more parties 202. The party-indicating data 202 may indicate what entities or entity roles may or may not participate in a secure room 104 generated from the compliance template 112. The party-indicating data 202 may include one or more specific entities (e.g., Healthcare Provider A, Pharmaceutical Company B, etc.). The party-indicating data 202 may include one or more entity roles (e.g., a hospital, a medical device company, a medical provider, etc.). The party-indicating data 202 may include a number of entities of each entity role that is allowed to participate in the secure room 104. In some embodiments, the party-indicating data 202 may include one or more specific entities or one or more entity roles that are prohibited from participating in the secure room 104. In some embodiments, whether a user 106 belongs to an entity of a certain entity role may be indicated by data included in that user's 106 account on the platform 102.

A compliance template 112 may include data indicating one or more secure room 104 functions or operations 204. The function-indicating data 204 may indicate what functions of the secure room 104 are available in the secure room 104 generated from the compliance template 104. The function-indicating data 204 may include data indicating what interaction methods are available in the secure room 104 (text chat, voice chat, etc.). The function-indicating data 204 may include data indicating whether files can be shared in the secure room 104, what types of files can be shared, etc. The function-indicating data 204 may include data indicating whether users 106 in the secure room 104 can view data about each other, and if so, how much or what specific pieces of data.

A compliance template 112 may include data indicating one or more interaction rules 206. An interaction rule 206 may include data that governs one or more interactions between the parties or users 106 indicated in the party-indicating data 202. An interaction rule 206 may include data indicating whether a certain type of interaction or whether a certain condition regarding parties or users 106 interacting is prohibited, allowed, or required.

As an example, a compliance template 112 may include party data 202 indicating that a hospital entity-role user 106 and a pharmaceutical company entity-role user 106 can participate in the secure room 104. The secure room functionality 204 may allow the users 106 to know what role each user 106 is, but not the specific identity of any user 106. The secure room functionality 204 may allow the users 106 to communicate through text chat and file-sharing, but not through voice chat or video chat. The interaction rules 206 may indicate that any healthcare data shared between the users 106 must be anonymized. The interaction rules 206 may indicate that a moderator 118 must review and approve all communications between the users 106 in the secure room 104 prior to the communications being available for view by the users 106 in the secure room 104.

The use of a compliance template 112 may allow the system 100 to quickly set up a secure room 104 where users 106 can communicate and collaborate in a compliant manner. There may be no immediate need to change a compliance template 112 if the compliance template 112 reflects and properly supports the intended interaction between the users 106. In cases where the intended interactions are not reflected by an existing compliance template 112 or where the intended interaction differs slightly from an existing compliance template 112, an existing compliance template 112 may be duplicated and altered to support the intended interaction. In such an embodiment, one or more entities associated with the system 100 may review the newly created compliance template 112 and validate it for compliance with laws and regulations. An entity reviewing the new compliance template 112 may include a user 106, a moderator 118, an auditor 120, an entity that owns, operates, or controls, the platform 102, or some other entity. The platform 102 may include a separate workflow for such review of the new compliance template 112.

Some components of the platform 102 may include dependencies on each other and may work in orchestration to provide the implementation of a secure room 104. In some embodiments, changing a component of the platform 102 or adjusting a parameter of the platform 102 may have an influence on the remaining components. A comprehensive view of a secure room 104 may help maintain consistent compliance overtime. The platform 102 may use user 106 roles and compliance templates 112 to drive a compliance barrier or the need for a specific attestation.

In some embodiments, the platform 102 may use a compliance template 112 to generate a technical setup 114. A technical setup 114 may include a data object that the platform 102 uses to set up a secure room 104 based on data in a compliance template 112 and data on the platform 102. In some embodiments, the parties data 202, secure room functionality data 204, and the interaction rules 206 of a compliance template 112 may form a framework for a secure room 104, and the platform 102 may use the framework to generate the components of the technical setup 114, which the platform then uses to implement the secure room 104.

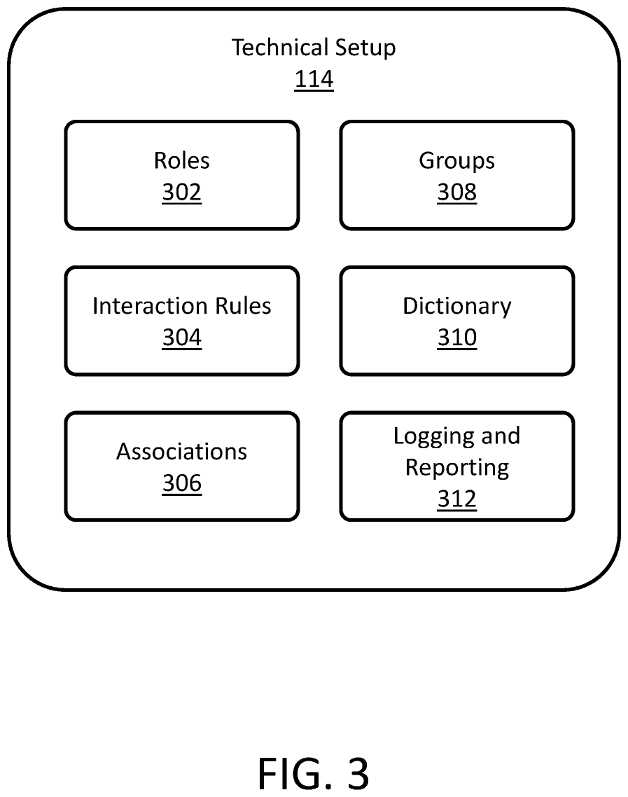

FIG. 3 depicts one embodiment of a technical setup 114. The technical setup 114 may include roles data 302, interaction rules data 304, association data 306, groups data 308, dictionary data 310, or logging and reporting data 312.

In one embodiment, the technical setup 114 may include roles data 302. The roles data 302 may include data about one or more roles a user 106 may take on. A role may be associated with one or more permissions or access patterns on the platform 102 or the secure room 104. As an example, a role may include a healthcare provider, a researcher, or a pharmaceutical company. In some embodiments, the platform 102 may include one or more standard roles that a user can take on. Standards role may reflect typical actors in the healthcare market. To reflect the real world set up, the platform 102 may duplicate and adjust one or more roles to help comply with legal requires and user 106 preferences. The platform 102 may adjust roles as well as compliance templates 112, as these components may be interrelated. In some embodiments, the platform 102 may log the creation of roles and adjustment of roles. In some embodiments, the platform 102 may use the party-indicating data 202 from the compliance template 112 and role data stored on the platform 102 to generate the roles data 302 of the technical setup 114.

In one embodiment, the technical setup 114 may include interaction rules data 304. The interaction rules data 304 may include data or computer-executable instructions that govern one or more interaction in a secure room 104. An interaction rule may include data or computer-executable instructions indicating whether a certain type of interaction or whether a certain condition in a secure room 104 is prohibited, allowed, or required. An interaction rule may include data or computer-executable instructions indicating that a certain user 106 may only send interaction data in the secure room 104 but cannot receive data. An interaction rule may include data or computer-executable instructions indicating that a certain user 106 may only receive interaction data in the secure room 104 but cannot send data. An interaction rule may include data or computer-executable instructions indicating that a user cannot view or download certain content in the secure room 104. An interaction rule may include data or computer-executable instructions indicating that only a certain user can view or download certain content. An interaction rule may apply to a user 106 based on that specific user 106 (i.e., the interaction rule only applies to that user 106 of the platform and no other users 106), based on a role the user 106 has, based on a role the user 106 has in that secure room 104, or based on other user 106 account data. In some embodiments, the secure room 104 may determine which content in the secure room 104 a user 106 can view or download based on the metadata associated with the content or the user 106 that generated or provided the content.

In one embodiment, an interaction rule may include an interaction rule that the secure room 104 requires a certain user 106 role. For example, an interaction rule may require that the secure room 104 include a user 106 with a hospital role. An interaction rule may include a rule that the secure room 104 prohibits a use 106 with a certain role. For example, an interaction rule may require that the secure room 104 not include a user 106 with a medical device company role. An interaction rule may include that a user 106 with a hospital role cannot know the identity of a user 106 with a life sciences company role. An interaction rule may include that a certain user 106 can only interact with a specific user 106 or a user 106 with a certain role.

In one or more embodiments, a moderator 118 may enforce an interaction rule. A moderator 118 may enforce a rule by preventing an interaction in the secure room 104. A moderator 118 may enforce an interaction rule by detecting a violation of the rule and performing a corrective action. In some embodiments, the platform 102 may automatically enforce an interaction rule. In some embodiments, the interactions rules data 304 may be based on the interactions rules 206 of the compliance template 112 or other data on the platform 102.

One benefit of an interaction rule-based execution within a secure room 104 is that interaction rules may be implemented easily in a machine-readable format, and as such, may allow for a high grade of automation within the overall system 100. In addition, interaction rules may allow for implementation of strong logging and reporting. In some embodiments, the platform 102 may be capable of auditing compliance with interaction rules and may be capable of interaction rules enforcement on a platform 102-wide or user device-specific scale.

In some embodiments, the technical setup 114 may include association data 306. The association data 306 may include data that may determine the relationship between one or more roles. These relationships may be on a spectrum from completely blinded to completely open. Similarly, the association data 306 may reflect that spectrum and may be implemented by the platform 102 such that the secure room 104 based on the technical setup 114 may allow for certain interactions between users 106 but may restrict other interactions, as determined by the association data 306.

In one embodiment, the technical setup 114 may include groups data 308. The groups data 308 may include data that may logically place one or more users 106 into one or more groups. A group may include one or more users 106 with the same role. A group may include one or more users 106 selected by one or more users of the platform 102. A group may be based on at least some of the association data 306. The groups data 308 may allow for predefined communication. In one example, a functional and business group may include one or more users 106 with data supplier roles for the discussion of treatment patterns and best practices in patient care-paths. In addition to functional and business groups, the groups data 308 may include a group of users 106 that may already have a legal foundation for interactions and, therefore, can communicate freely or within certain boundaries.

In some embodiments, the technical setup 114 may include dictionary data 310. The dictionary data 310 may include one or more entries. An entry may include a key and one or more values associated with the key. The key may include one or more words, phrases, or other portions of text or speech. The one or more values may include a replacement word, phrase, or other portion of text or speech or other data. The key of a dictionary data 310 entry may be black- or whitelisted in interactions between users 106. The interactions rules 304 data may include a responsive action that is triggered in response to a user 106 using a word or phrase that is a key. The responsive action may include deleting the instance of the word or phrase in the interaction, replacing the instance of the word or phrase in the interaction with another word or phrase as indicated by an associated value in the entry, obscuring the instance of the word or phrase in the interaction, or some other responsive action. The responsive action may occur prior to the interaction data being made viewable or accessible by other users 106 of the secure room 104.

For example, the dictionary data 310 may include an entry where the brand name of a specific drug is the key and the one or more values associated with the key may include a generic name for the drug. A healthcare provider user 106 may use text chat in a secure room 104 to send a text chat message to another user 106. The text chat message may include the brand name of the drug. In response, the platform 102 may replace the brand name in the text chat message with a generic name (as provided by the dictionary data 310) for that drug so that the other user 106 (e.g., a life sciences sponsor) may remain blinded to the healthcare provider user 106. In some embodiments, the platform 102 may support tagging based on the dictionary data 310. For example, in the previous example, the platform 102 may also tag the text chat message that includes the brand name of the drug so that the text chat message can be quickly located for audit or other purposes.

In one or more embodiments, an AI model (such as an AI moderator 118) may detect the presence of a key of the dictionary data 310 in interaction data. The AI model may replace the key in the interaction data with replacement data, or may perform another responsive action based on the interaction rules of the secure room 104.

In one embodiment, the technical setup 114 may include logging and reporting data 312. The platform 102 may track or report one or more interactions between users 106 in a secure room 104 or other events that occur in a secure room 104 based on the logging and reporting data 312. Logging interactions or events may include logging contributions to or interactions in a secure room 104, monitored or restricted near real-time synchronous communication (e.g., text chat), the downloading of files, or data creation. The reporting may include general or targeted reporting of one or more secure room 104 interactions or events as determined by the technical setup 114. These compliance reports can, in some embodiments, serve as a foundation for federal- or industry-specific platform 102 reviews to help ensure legality or ethical set up of interactions.

In some embodiments, the platform 102 may be in data communication with a distributed ledger 122. For example, in some embodiments, the computing device hosting the platform 102 or the platform 102 may host a distributed ledger node that is associated with the distributed ledger 122. In other embodiments, the platform 102 may be in data communication with the distributed ledger node. The platform 102 may generate a distributed ledger transaction and send the transaction to the distributed ledger node in order for the distributed ledger node to add the transaction to the distributed ledger 122. In some embodiments, the platform 102 may send the data to be recorded to the distributed ledger node, and the distributed ledger node may generate the distributed ledger transaction.

In one embodiment, the platform 102 may record one or more interactions or events to the distributed ledger 122. Such interactions or events may include the creation of an illegal compliance template 112 or technical setup 114 or the generation of a secure room 104 based on an illegal compliance template 112 or technical setup 114. Such interactions or events may include one or more interactions, communications, or collaboration events that occurred in a secure room 104. The writing of such interactions or events to the distributed ledger may provide immutability of records related to such interactions or events.

In some embodiments, the logging and reporting functionality of the platform 102 based on the logging and reporting data 312 may assist with audit functionality on the platform 102. Governmental and other legal bodies, as auditors 120, may gain access, through the platform's 102 audit functionality, to selected logs or reports and may be associated with an investigated scenario. The scenario may include a clear compliance and legal justification. The scenario may define the users 106, the time frame, or the content in question. The platform 102 may grant the auditor 120 access to related distributed ledger 120 entries for event verification or case analysis. The selected provisioning of logging or reporting data to external parties may prevent random forum analysis and may help the neutral and de-identified event capturing and processing for compliance and security reasons.

As mentioned above, one or more users 106 may generate a compliance template 112 from which an instance of a technical setup 114 may be created. The platform 102 may generate the technical setup 114 based on the compliance template 112. The compliance template 112 and the technical setup 114 may depend on each other with regards to functionality and rationale. In some embodiments, the one or more users 106 may generate the technical setup 114 without first creating the compliance template 112, and the platform 102 may generate the secure room 104 based on the technical setup 114. In another embodiment, the platform 102 may generate the secure room based on the compliance template 112 without generating a technical setup 114.

In one embodiment, a change in the compliance template 112 may cause the platform 102 to change the corresponding technical setup 114. The platform 102 may change the technical setup 114 automatically and in real-time or near real-time. The compliance template 112 may be driven by the need for compliance against rules and regulations laid out by federal bodies and states. Therefore, one or more changes in laws and regulations may lead to a need for change in the compliance template 112 and, consequently, the technical setup 114. As laws and regulations are subject to regular change and adjustments, the concept of compliance template 112 and technical setup 114 versioning may help in implementing and modifying a secure room 104 to align with the technical setup 114.

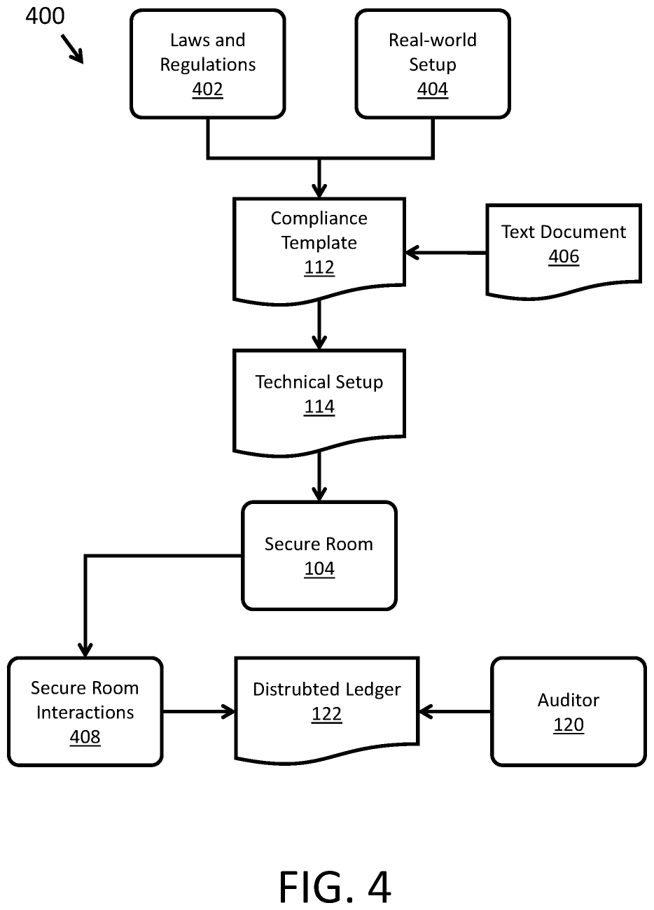

FIG. 4 depicts one embodiment of a flow 400 that shows an example interaction that may happen from the initial phase of generating a compliance template 112 to the implementation and use of a corresponding secure room 104. Based on laws and regulations 402 in the real world, set up of a compliance template 112 may be selected as a foundation for a collaboration between users 106. The compliance template 112 may describe, in clear language, the proposed collaboration compliance and legal guidance, participating partners, or technology to implement. The actual technical implementation of the secure room 104 may be derived from the technical setup 114, which may be based on the compliance template 112. One or more interactions 408 in the secure room 104 may be logged in a distributed ledger 122 and, therefore, may be ready for audit purposes and general logging and reporting.

In one embodiment, the platform 102 may determine which secure rooms 104 a user 106 may participate in based on permissions data. In some embodiments, the permissions data 502 may include rules, conditional statements, or other logical structures that may determine which secure rooms 104 the user 106 may join. A restricted and blinded user 106 on the platform 102 may have been vetted by the attestation functionality of the platform 102. As the configuration of a secure room 104 may be the result of a compliance template 112 or technical setup 114, a restricted, blinded user 106 might participate in a variety of secure rooms 104 where one or more secure rooms 104 may be based on different compliance templates 112 or technical setups 114 for specific participants and legal requirements.

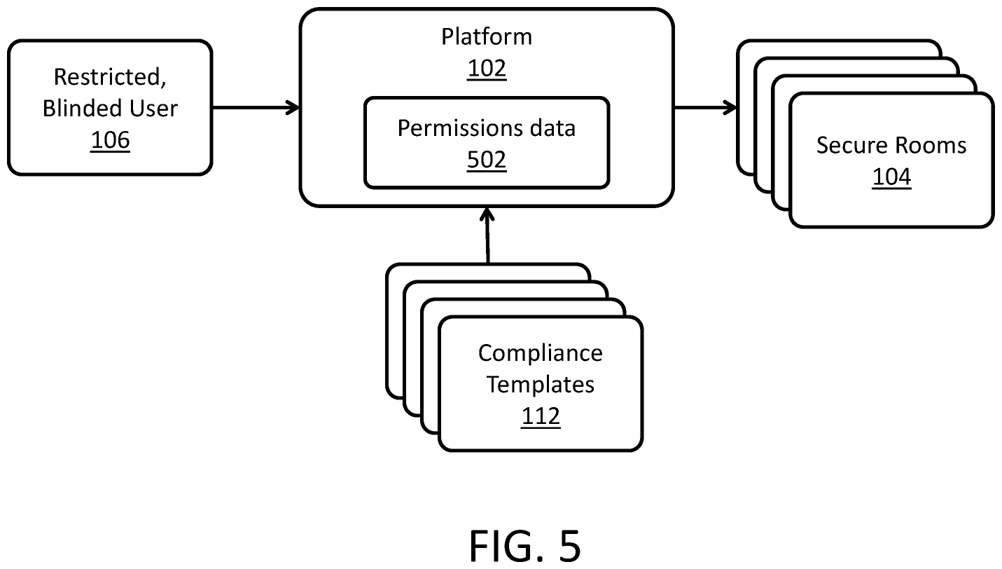

FIG. 5 depicts one embodiment of user 106-secure room 104 associations. As can be seen in FIG. 5, a restricted, blinded user 106 may log onto the platform 102 in order to joint a secure room 104. The platform 102 may determine, from the permissions data 502 associated with the platform 102, which secure rooms 104 the user 106 may joint. The permissions data 502 may be derived from one or more compliance templates 112 that the one or more secure rooms 104 may be based on. The permissions data 502 may be derived from one or more technical setups 114 that the one or more secure rooms 104 may be based on.

In one embodiment, the platform 102 may obtain roles data, associations data, or groups data associated with the user's 106 account on the platform. The platform 102 may determine, using the permissions data 502, whether the user's role data, associations data, or groups data is compatible with a secure room's parties data 202 of the compliance template 112 that the secure room 104 is based on, or is compatible with the 104 roles data 302, associations data 306, or groups data 308 of the technical setup 114 the secure room 104 is based on. For example, the parties data 202 may include a list of users 106, and in response to the user 106 being included in that list of users 106, the user 106 may be able to participate in the secure room 104. The roles data 302 may include one or more roles, in response to the user's 106 account data indicating that the user 106 has a role in the roles data 302, the user 106 may be able to participate in the secure room 104. The groups data 308 may include a group of users 106, and in response to the user's 106 account data indicating the user 106 in in the group, the user 106 may be able to participate in the secure room 104.

In some embodiments, a user 106 may have different options, functionality, or user interface components available in a secure room 104 based on the technical setup 114 of the secure room 104. A user 106 may face a highly restricted secure room 104 for a certain topic in one secure room 104, and the same user 106 might be free to communicate openly with other users 106 in a different secure room 106 based on a different technical setup 114. The technical implementation and graphical user interface of the secure room 104 may be designed to accurately reflect the current technical setup 114 up in the user's 106 current secure room 104.

In one embodiment, the platform 102 may perform logging and reporting functionality that relates to a specific user 106 and with a specific context of a secure room 104 and role. The platform 102 may maintain confidentiality of user 106 actions in secure rooms 104 that are not related to a specific reporting need. As an example, the reporting of a secure room 104 for life science users 106 where all communication is open may be separated from the reporting of a secure room 104 between a life science user 106 and a healthcare provider user 106.

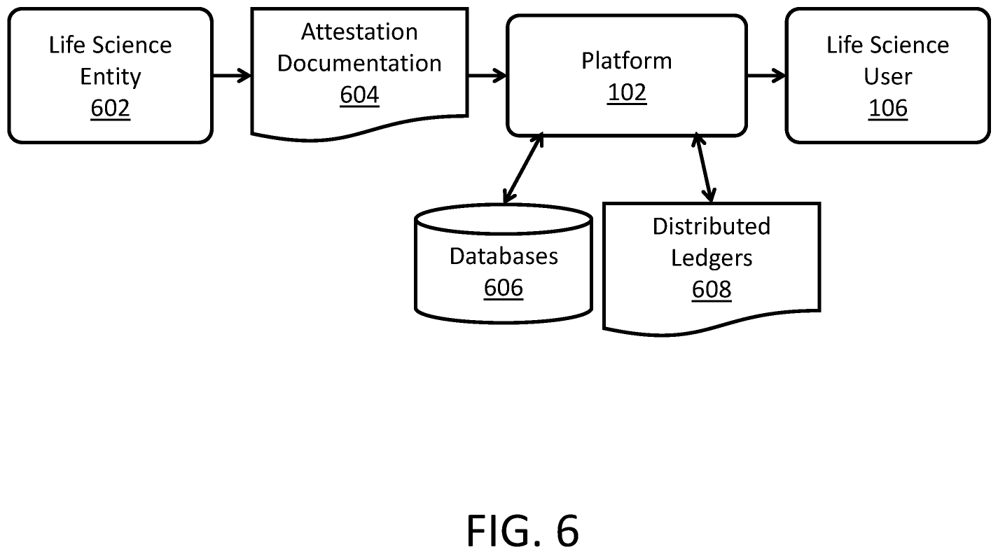

FIG. 6 depicts one embodiment of the onboarding process of a life science entity 602 into a secure room 104. As part of the onboarding process, the life science entity 602 may submit attestation documentation 604 to the platform 102. The platform 102 may use the attestation documentation 604 to validate the identity of the life science entity 602 and determine what roles, groups, associations, and other data should be associated with the life science entity's user 106 account such that the life science entity 602 may participate in one or more secure rooms 104 in a compliant manner. The attestation documentation 604 may include documents identifying the life science entity. Such identification documents may include a government ID card, a diploma, government credential information, corporate filing documents, or other documents or information that may identify the life science entity 602 and may indicate what permissions the life science entity should have on the platform 102. The platform 102 may verify the attestation documentation 604. Verifying the attestation documentation 604 may include checking the information in the attestation documentation 604 against the information stored in one or more databases 606 or one or more distributed ledgers 608. The one or more databases 606 may include a government database, a university database that includes graduation information, a state board of medication database, a secretary of state business entity database, or other similar databases. The one or more distributed ledgers 608 may include a distributed ledger that tracks governmental, university, or other information.

Based on the attestation findings, the platform 102 may generate a user 106 account for the life science entity 602 on the platform 102. The user 106 account may include roles data, associations data, groups data, or other permissions data that the platform 102 may use to determine which secure rooms 104 the user 106 may join, and what functionality within the secure room 104 the user 106 may perform. Such permissions may restrict and blind the user 106 to represent the life science entity 602 on the platform 102 and in specific secure rooms 104.

In some embodiments, a user 106 may submit attestation documentation 604 to the platform 102 after the user's 106 account on the platform 102 has been generated. This attestation documentation 604 may include data indicating that the user 106 agrees to comply with one or more interactions rules data 304 of a secure room 104, data indicating the user's 106 interactions in a secure room 104 will or will not contain certain data, or other attestation information. The user 106 may submit the attestation documentation 604 in response to attempting to perform an action in a secure room 104 where that action requires certain attestation from the user 106. For example, in one embodiment, the interaction rules data 304 for a secure room 104 may indicate that a user 106 must attest that the user's 106 communications in the secure room 104 will not contain marketing data. In response to the user 106(1) attempting to send a text chat message to another user 106(2) in the secure room 104, the platform 102 may prompt the user 106(1) to attest that the user 106(1) will not send marketing data. The platform 102 may prompt the user 106 to provide required attestation documentation 604 in response to logging onto the platform 102, attempting to enter a secure room 104, while in a secure room 104, or at some other point in time.

In some embodiments, one or more moderators 118 may work assist with compliance on the platform 102. This moderation function may be two-fold. On the one hand, a moderator 118 may review or approve interactions in a secure room 104 as they are tagged and filtered by a dictionary function based on the dictionary data 310 associated with the secure room's 104 technical setup 114. A moderator 118 may flag outliers and incorrectly tagged interaction data and may send such interaction data to the user 106 that generated the interaction data for review. On the other hand, a moderator 118 may observe and potentially restrict interactions in a secure room 104. Whether a certain secure room 104 interaction, such as access to a dataset 108 or an insight 110, or communications (text chat, video chat, etc.) is compliant may be determined based on the data of the technical setup 114 or other platform 102 parameter settings.

As an example, the platform 102 may generate a secure room 104 based on a compliance template 112 that describes a relationship between Pharma XYZ and Dermatology Partner 123, where Pharma XYZ has access to a purchased data offering (Plaque Psoriasis 2018) from Dermatology Partner 123. A user 106(1) named Kim from Pharma XYZ with the role “Market Access User” may use the platform 102 to enter the secure room 104 to view and download the dataset 108 that includes the Plaque Psoriasis 2018 data offering. In this example, in order to view or download the data offering, the interaction rules 304 may require Kim (user 106(1)) to submit attestation documentation 604. No user 106 from Dermatology Partner 123 may have entered the secure room 104. Kim (user 106(1)) may digitally sign the attestation documentation 604 about data usage. The platform 104 may digitally timestamp and store Kim's attestation documentation 604 (e.g., store the attestation documentation 604 on the distributed ledger 122 so that the attestation documentation is stored immutably). Kim may then be able to download the data offering to her user device and perform some work outside of the platform 102.

Continuing the example, after performing some analysis, Kim (user 106(1)) may have some questions about some derived insight from the Plaque Psoriasis 2018 data offering. Kim may enter the same secure room 104 associated with the Plaque Psoriasis 2018 data offering. Kim may generate a secure message as part of some communication data in the secure room 104, and the secure message may include her questions that reference the dataset 108. Kim may submit the message to the secure room 104 so that one or more other users 106(2) may view it. In this example, the interaction rules data 304 of the secure room 104 may require Kim to submit additional attestation documentation 604 to send the message. The attestation documentation 604 may include language agreeing that the message relates to the data in the secure room 104 and does not include content that would constitute a marketing or sales offer. In response to Kim submitting the attestation documentation 604 and digitally signing it, the platform 102 may digitally timestamp and store the attestation documentation 604. The platform 102 may also hash, digitally timestamp, and store Kim's message for compliance and audit purposes.

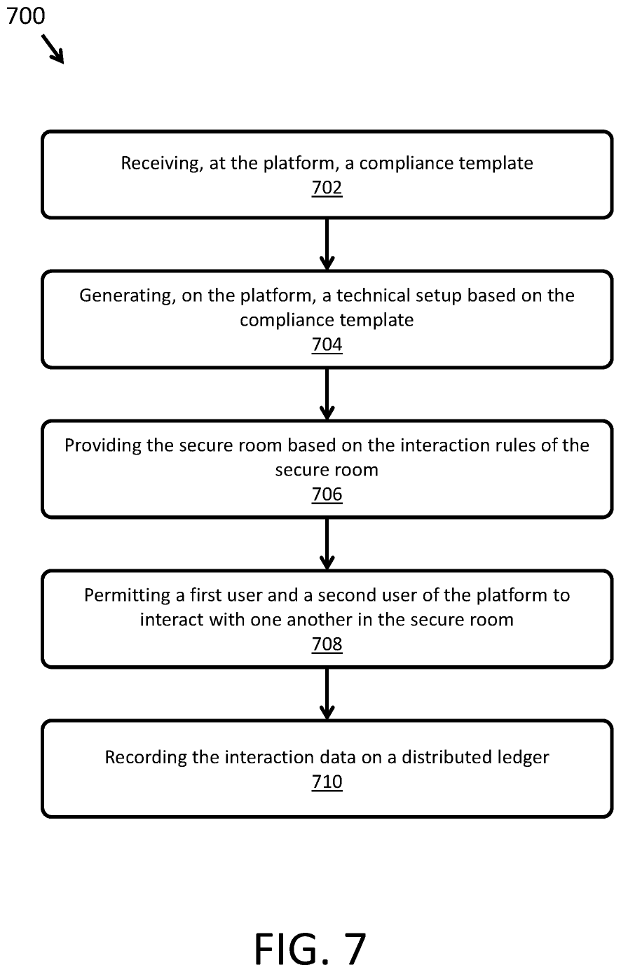

FIG. 7 depicts one embodiment of a method 700. The method 700 may include a computer-implemented method for compliant interaction between parties in a secure room 104 implemented on a compliance and insight platform 102. The method 700 may include receiving, at the platform 102, a compliance template 112 (step 702). The compliance template 112 may include data indicating one or more entities or parties 202, functionality 204 of the secure room 104, and interaction rules 206 of the secure room 104. The method 700 may include generating, on the platform 102, a technical setup 114 based on the compliance template 112 (step 704). The technical setup 114 may include computer-executable instructions configured to enforce the interaction rules 304 in the secure room 104. The method 700 may include providing the secure room 104 based on the interactions rules 304 of the secure room 104 (step 706). The method 700 may include permitting a first user 106(1) and a second user 106(2) of the platform 102 to interact with one another in the secure room 104 (step 708). The first user 106(1) and the second user 106(2) interacting in the secure room 104 may include the first user 106(1) sending interaction data to the secure room 104, and the platform 102 making at least a portion of the interaction data viewable by the second user 106(2). The method 700 may include recording the interaction data on a distributed ledger 122 (step 710).

In some embodiments, the steps 702-710 of the method 700 may be performed by one or more components of the system 100 or platform 102 as has discussed herein. The method 700 may include other steps or functionality discussed herein.

In one embodiment, one or more of the components of the systems and methods disclosed herein may include a data object. For example, a compliance template 112 may include a compliance template data object that may include data associated with the compliance template 112 and functions that the compliance template 112 may carry out. Similarly, a secure room 104 may include a data room data object that may include data associated with the secure room 104 and may include functions that the secure room 104 may perform. Other data objects may include user 106 data objects, interaction data data objects, moderator 118 data objects, or other data objects corresponding to components.

In some embodiments, the platform 102 may include various modules that may perform one or more functions of the platform 102. A module may include hardware, software, or a combination of hardware and software. For example, the platform 102 may include a compliance module that may execute one or more interactions rules in the one or more secure rooms 104. The platform 102 may include a secure room module that may implement the one or more secure rooms 104. The platform 102 may include an attestation module that may perform the attestation process for onboarding a user 106 into a secure room 104 or allow a user to perform certain interactions in a secure room 104. The platform 102 may include a logging and reporting module that may tag interaction data, store interaction data, or other logging and reporting functionality.

The systems and methods disclosed herein present several technical advantages over prior art and conventional efforts to provide compliant healthcare data sharing in a compliant manner. In contrast to a conventional one-off setup, one or more entities desiring to share healthcare data may review the setup of the secure rooms 104 and the related compliance templates 112 and technical setups 114 once and approve them once on each side. An entity's information technology (IT) department may examine the distributed ledger 122-based logs for legal and compliance requirements. In case of an audit by an auditor 120, the distributed ledger 122-based logs can easily be extracted and filtered for selected collaboration in focus.

Furthermore, comparing the conventional collaboration approach for pharmaceutical companies and healthcare providers with the secure room 104 collaboration of the present systems and methods leads to the following conclusions. The use of a secure room 104 may provide a standardized and expedited path towards collaboration or compliant interactions between pharmaceutical companies, healthcare providers, and other actors in the healthcare field. The systems and methods drastically reduce the burden on legal and IT departments to facilitate non-standardized collaboration efforts by using secure room 104 technology. The secure room 104 set up may allow for restricted, blinded users 106 who can collaborate, communicate, or work on insights towards patient lives' improvements without the ethical, legal, and compliance burden that is normally imposed on these actors.

In conventional technological collaboration setups, healthcare providers engage in data sharing and research by participating in registries, often sponsored by medical associations (e.g., the American Academy of Dermatology, the American College of Cardiology). These medical associations use the data to provide insights, benchmarks, and summary data back to providers and open opportunities for research and quality initiatives. In addition, these associations create patient deidentified data for life sciences and other research use in the form of registries. In most cases, the associations contract with or partner with a vendor to extract the data from providers and create the registry dataset. While this process usually meets regulatory requirements and blinds the provider to any influence by a data consumer, it also takes months to years for data to flow from a provider to a researcher. One limitation of these conventional setups is that data are no longer current (often 6 months to a year old) by the time analysis can be conducted. Another limitation is that there is no mechanism for a researcher to ask questions or better understand the data from the source. Finally, the data flows from the provider into a fixed data dictionary with no practical mechanism to extract more data from the provider or fix any errors. Utilization of a secure room 104 setup as disclosed herein, has a tremendous potential to expedite these processes.

While the present disclosure has discussed the systems and methods herein in relation to healthcare data, healthcare providers, pharmaceutical companies, and other healthcare-related concepts and entities, the systems and methods disclosed herein could be applied to other types of data and entities. The other types of data and entities could include data and entities that operate under a compliance or legal framework. Such data may include financial data, securities data, data brokerage data, scientific data, or other types of data.

While the making and using of various embodiments of the present disclosure are discussed in detail herein, it should be appreciated that the present disclosure provides many applicable inventive concepts that are embodied in a wide variety of specific contexts. The specific embodiments discussed herein are merely illustrative of specific ways to make and use the disclosure and do not delimit the scope of the disclosure. Those of ordinary skill in the art will recognize numerous equivalents to the specific apparatuses, systems, and methods described herein. Such equivalents are considered to be within the scope of this disclosure and may be covered by the claims.

Furthermore, the described features, structures, or characteristics of the disclosure may be combined in any suitable manner in one or more embodiments. In the description contained herein, numerous specific details are provided, such as examples of programming, software, user selections, hardware, hardware circuits, hardware chips, or the like, to provide understanding of embodiments of the disclosure. One skilled in the relevant art will recognize, however, that the disclosure may be practiced without one or more of the specific details, or with other methods, components, materials, apparatuses, devices, systems, and so forth. In other instances, well-known structures, materials, or operations may not be shown or described in detail to avoid obscuring aspects of the disclosure.

These features and advantages of the embodiments will become more fully apparent from the description and appended claims, or may be learned by the practice of embodiments as set forth herein. As will be appreciated by one skilled in the art, aspects of the present disclosure may be embodied as an apparatus, system, method, computer program product, or the like. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present disclosure may take the form of a computer program product embodied in one or more computer-readable media having program code embodied thereon.

In some embodiments, a module may be implemented as a hardware circuit comprising custom (very large-scale integration) VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in software, the program code may be stored and/or propagated on in one or more computer-readable media.

In some embodiments, a module may include a smart contract hosted on a blockchain. The functionality of the smart contract may be executed by a node (or peer) of the blockchain network. One or more inputs to the smart contract may be read or detected from one or more transactions stored on or referenced by the blockchain. The smart contract may output data based on the execution of the smart contract as one or more transactions to the blockchain. A smart contract may implement one or more methods or algorithms described herein.

The computer program product may include a computer-readable storage medium (or media) having computer-readable program instructions thereon for causing a processor to carry out aspects of the present disclosure. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer-readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform aspects of the present disclosure.

Aspects of the present disclosure are described herein with reference to flowchart illustrations or block diagrams of methods, apparatuses, systems, algorithms, or computer program products according to embodiments of the disclosure. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The schematic flow chart diagrams included herein are generally set forth as logical flow chart diagrams. As such, the depicted order and labeled steps are indicative of one embodiment of the presented method. Other steps and methods may be conceived that may be equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

The schematic flowchart diagrams and/or schematic block diagrams in the Figures illustrate the architecture, functionality, and operation of possible implementations of apparatuses, systems, methods and computer program products according to various embodiments of the present disclosure. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

Thus, although there have been described particular embodiments of the present disclosure of a new and useful COMPLIANT INSIGHT FORUM AND ANALYSIS FOR HEALTHCARE, it is not intended that such references be construed as limitations upon the scope of this disclosure.

What is claimed is:

1. A system for compliant interaction between parties, comprising:
a computing device hosting a compliance and insight platform executing on the computing device, wherein
the platform includes a first secure room hosted on the platform, wherein the first secure room includes a restricted environment that hosts interaction data between users based on one or more interactions rules of the secure room, and wherein the first secure room is configured to
receive first interaction data from a first user in the first secure room,
provide at least a portion of the first interaction data to a second user in the first secure room, and
blind and restrict the first user and the second user from each other's identity, and
the platform further includes a moderator of the first secure room, wherein the moderator is configured to moderate the interaction data in the first secure room.

2. The system of claim 1, wherein the first interaction data comprises at least one of:
text chat data;
voice chat data; or
video chat data.

3. The system of claim 1, wherein the first secure room is further configured to:
receive second interaction data from the second user, wherein the second interaction data includes a dataset; and
provide at least a portion of the second interaction data to the first user.

4. The system of claim 3, wherein the platform being configured to provide the at least a portion of the second interaction data to the first user includes the platform making the dataset available to download from the platform by the first user.

5. The system of claim 1, wherein:
the platform is further configured to generate the first secure room based on a first compliance template received by the platform; and
the first compliance template includes data indicating one or more entities, functionality of the first secure room, and data indicating one or more interaction rules.

6. The system of claim 5, wherein:
the platform is further configured to generate a first technical setup based on the first compliance template;
the first technical setup includes
data indicating one or more roles for each of the first user and the second user based on the data indicating one or more entities from the compliance template, and
the one or more interaction rules of the secure room based on the data indicating the one or more interaction rules from the compliance template; and
the platform generating the first secure room based on the first compliance template includes the platform generating the first secure room based on the first technical setup.

7. The system of claim 6, wherein:
the platform is further configured to generate a second secure room based on a second technical setup, wherein the second secure room is hosted on the platform; and
the data of the second technical setup indicating the roles and interaction rules of the second secure room are at least partially different than the data indicating the roles and interaction rules of the first secure room.

8. The system of claim 1, wherein the one or more roles include at least one of:
a pharmaceutical company; or
a healthcare provider.

9. The system of claim 1, wherein the moderator comprises at least one of:
a third user of the platform; or
an artificial intelligence model.

10. The system of claim 1, wherein:
the first secure room comprises dictionary data, wherein the dictionary data includes a plurality of entries, and wherein each entry of the plurality of entries includes at least one of a word or a phrase; and
in response to the first interaction data including an entry of the dictionary data, the platform is further configured to at least one of
remove, from the first interaction data, the entry, or
replace, in the first interaction data, the entry with one or more predetermined replacement words.

11. The system of claim 1, wherein:
the platform further comprises a distributed ledger node; and
the platform is further configured to
generate a first distributed ledger transaction, wherein the first distributed ledger transaction includes at least a portion of the first interaction data, and
add the first distributed ledger transaction to a distributed ledger associated with the distributed ledger node.

12. The system of claim 11, wherein the platform is further configured to:
receive attestation documentation from the first user; and
generate a second distributed ledger transaction based on the attestation documentation; and
add the second distributed ledger transaction to the distributed ledger associated with the distributed ledger node.

13. The system of claim 1, wherein the platform is further configured to:
prompt the first user to provide attestation documentation to the platform;
receive the attestation documentation from the first user; and
verify the attestation documentation against at least one of an external database or a distributed ledger.

14. The system of claim 13, wherein the platform is configured to prompt the first user to provide the attestation document in response to at least one of:
the first user attempting to join the first secure room; or
the first user attempting to provide the first interaction data to the platform.

15. The system of claim 1, wherein the platform is further configured to provide the first interaction data to an auditor user of the platform, wherein the platform being configured to provide the first interaction data to the auditor user includes at least one of:
the platform providing the auditor user access to a data storage of the platform, wherein the data storage stores the first interaction data; or
the platform providing the auditor user access to a distributed ledger, wherein the distributed ledger includes a distributed ledger transaction that includes the first interaction data.

16. A computer-implemented method for compliant interaction between parties in a secure room implemented on a compliance and insight platform, comprising:
receiving, at the platform, a compliance template, wherein the compliance template includes data indicating one or more entities, functionality of the secure room, and interaction rules of the secure room;
generating, on the platform, a technical setup based on the compliance template, wherein the technical setup includes computer-executable instructions configured to enforce the interaction rules in the secure room;
providing the secure room based on the interactions rules of the secure room;
permitting a first user and a second user of the platform to interact with one another in the secure room, wherein the first user and the second user interacting in the secure room includes
the first user sending interaction data to the secure room, and
the platform making at least a portion of the interaction data viewable by the second user; and
recording the interaction data on a distributed ledger.

17. The computer-implemented method of claim 16, wherein the interaction data comprises at least one of:
text chat data;
voice chat data; or
video chat data.

18. The computer-implemented method of claim 16, further comprising:
detecting, via an artificial intelligence model of the platform, the presence of a first predetermined word in the interaction data; and
replacing, via the artificial intelligence model, the first predetermined word in the interaction data with a second predetermined word prior to the platform making the at least a portion of the interaction data viewable by the second user.

19. The computer-implemented method of claim 16, further comprising:
prompting the first user to provide attestation documentation to the platform;
receive, at the platform, the attestation documentation from the first user; and
recording at least a portion of the attestation documentation on the distributed ledger.

20. A non-transitory computer-readable storage medium having a first set of computer-executable instructions stored thereon, wherein in response to a computer processor executing the computer-executable instructions, the computer processor is configured to:
receive, at a compliance and insight platform, a compliance template for a secure room, wherein the compliance template includes data indicating one or more entities, functionality of the secure room, and interaction rules of the secure room;
generate, on the platform, a technical setup based on the compliance template, wherein the technical setup includes a second set of computer-executable instructions configured to enforce the interaction rules in the secure room;
provide the secure room based on the interaction rules of the secure room;
permit a first user and a second user of the platform to interact with one another in the secure room, wherein the first user and the second user interacting in the secure room includes
the first user providing a dataset to the secure room,
the platform making at least a portion of the dataset downloadable from the platform by the second user,
the second user sending text-based communication data to the secure room, and
the platform making at least a portion of the text-based communication data viewable in the secure room by the first user; and
record the text-based communication data on a distributed ledger.
